(19) United States
(12) Patent Application Publication
(10) Pub. No.: US 2013/0012775 A1
(43) Pub. Date: Jan. 10, 2013

(54) TRANSMITTING AND RECEIVING APPARATUS, ANTENNA UNIT, AND BODY-INSERTABLE APPARATUS SYSTEM

(75) Inventor: Toru MIYAZONO, Tokyo (JP)

(73) Assignee: OLYMPUS MEDICAL SYSTEMS CORP., Tokyo (JP)

(21) Appl. No.: 13/551,777

(22) Filed: Jul. 18, 2012

Related U.S. Application Data
(63) Continuation of application No. PCT/JP2011/076912, filed on Nov. 22, 2011.

(30) Foreign Application Priority Data
Nov. 29, 2010 (JP) 2010-265758

(51) Int. Cl. A61B 1/04; H01Q 1/00

(52) U.S. Cl. 600/109; 343/720

(57) ABSTRACT

A transmitting and receiving apparatus that is connected with each of plural connection targets, which include an antenna unit and a charger, to perform transmission/reception of signals. The transmitting and receiving apparatus includes a connection portion that is provided on a surface of a reception main body unit of the transmitting and receiving apparatus, connectable with the antenna unit and another connection target, and is connected, at one time, with only one connection target to prevent a malfunction.

### CROSS REFERENCES TO RELATED APPLICATIONS

This application is a continuation of PCT international application Ser. No. PCT/JP2011/076912 filed on Nov. 22, 2011 which designates the United States, incorporated herein by reference, and which claims the benefit of priority from Japanese Patent Applications No. 2010-265758, filed on Nov. 29, 2010, incorporated herein by reference.

### BACKGROUND OF THE INVENTION

1. Field of the Invention

The present invention relates to a transmitting and receiving apparatus which receives, using an antenna outside a subject, wireless signals transmitted from, for example, a capsule endoscope that has been introduced into the subject, an antenna unit, and a body-insertable apparatus system.

2. Description of the Related Art

Conventionally, endoscopes have been widely used as a medical observation apparatus that is introduced into the body of a subject such as a patient to thereby observe the body cavity. In addition, in recent years, a swallowable endoscope (a capsule endoscope) which includes, inside a capsule casing, an imaging device and a communication device that transmits image data imaged by the imaging device to the outside of the body in a wireless manner has been developed. The capsule endoscope is swallowed by the mouth of a patient for observation of the body cavity and sequentially images internal organs such as the esophagus, stomach, and small intestine, while moving inside the internal organs according to their peristaltic motions until the capsule endoscope is naturally excreted from the body.

Image data that has been imaged inside the body cavity by the capsule endoscope, while the capsule endoscope is moved inside the body cavity, is sequentially transmitted to the outside of the body by wireless communication, and stored in a memory device installed inside or outside a transmitting and receiving apparatus provided outside the body or image-displayed on a display installed in the transmitting and receiving apparatus. On the doctor or nurse side, the image data stored in the memory device is imported to a workstation through a cradle in which the transmitting and receiving apparatus is inserted, so that a diagnosis is made on the basis of an image displayed on a workstation display or an image displayed upon reception on a display installed in the transmitting and receiving apparatus.

However, when the transmitting and receiving apparatus is inserted into the cradle in a state in which a reception antenna is connected, the cradle becomes unstable due to a change in the center of gravity of the cradle by a cable or the like that is connected to the reception antenna, and the transmitting and receiving apparatus may be detached from the cradle while the stored image data is imported to the workstation from the transmitting and receiving apparatus.

With regard to this problem, for example, in Japanese Laid-open Patent Publication No. 2007-081492, a received information transmitting system that includes a reception unit that is attachable to and detachable from an antenna unit that receives information such as image data, and a cradle having a protrusion for enabling connection only when the antenna unit is disconnected, and stably transmits information in a state in which the reception unit and the cradle are connected to each other while the reception unit is not attached to the antenna unit has been disclosed. In addition, the reception unit of the received information transmitting system may be exclusively connected with the antenna unit and the cradle.

In the conventional cradle described above, a process of the reception unit connected to the cradle, for example, a transmission process of information, a charging process of the reception unit, or the like is electrically switched by switching of a signal within the cradle.

### SUMMARY OF THE INVENTION

A transmitting and receiving apparatus according to an aspect of the invention is connected with each of a plurality of connection targets, which include an antenna unit and a charger, to perform transmission/reception of a signal, and includes: a connection portion that is provided on a surface of the transmitting and receiving apparatus, connectable with the plurality of connection targets, and is connected, at one time, with only one connection target to prevent a malfunction.

An antenna unit according to another aspect of the invention is connectable with a plurality of connection targets, which include a charger, includes a connection portion that is connected, at one time, with only one connection target to prevent a malfunction, and is connectable with a transmitting and receiving apparatus that performs transmission/reception of a signal, the antenna unit including: a connector that is connectable with a display device for displaying image data received by the antenna unit, the connector being provided on a surface of the antenna unit that does not oppose to the transmitting and receiving apparatus when the antenna unit is mounted on the transmitting and receiving apparatus.

A body-insertable apparatus system according to another aspect of the invention includes: a body-insertable apparatus that is introduced into a subject and moves inside the subject; and a transmitting and receiving apparatus that transmits/receives a signal to/from another connection target including the body-insertable apparatus and a charger, wherein the transmitting and receiving apparatus includes an antenna unit that receives information from the body-insertable apparatus in a wired or wireless manner, and a reception main body unit that includes a first connection portion that is attachable to and detachable from the antenna unit and a second connection portion that is provided on a surface, which is covered by the antenna unit when the antenna unit is mounted, and is connectable with another connection target including the charger.

The above and other features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a schematic diagram illustrating an overall configuration of a body-insertable apparatus system according to a first embodiment of the invention;

FIG. 2 is a block diagram illustrating a configuration of a transmitting and receiving apparatus shown in FIG. 1;

FIG. 3 is a perspective diagram schematically illustrating a configuration of the transmitting and receiving apparatus of the body-insertable apparatus system according to the first embodiment of the invention;

FIG. 4 is a schematic diagram illustrating a configuration of a main portion of the body-insertable apparatus system according to the first embodiment of the invention;

FIG. 5 is a schematic diagram illustrating a configuration of a main portion of a body-insertable apparatus system according to a modified example 1 of the first embodiment of the invention;

FIG. 6 is a perspective diagram schematically illustrating a configuration of a main portion of a body-insertable apparatus system according to a modified example 2 of the first embodiment of the invention;

FIG. 7 is a perspective diagram schematically illustrating a configuration of a transmitting and receiving apparatus of a body-insertable apparatus system according to a second embodiment of the invention;

FIG. 8 is a schematic diagram illustrating a configuration of a main portion of the body-insertable apparatus system according to a second embodiment of the invention; and

FIG. 9 is a perspective diagram schematically illustrating a configuration of a main portion of a body-insertable apparatus system according to a modified example of the second embodiment of the invention.

### DETAILED DESCRIPTION OF THE PREFERRED EMBODIMENTS

Hereinafter, embodiments of the invention will be described in detail with the accompanying drawings. Incidentally, the invention is not limited to the following embodiments. In the following description, each drawing merely schematically shows shapes, sizes, and positional relationships in a degree such that contents of the invention can be understood, and therefore the invention is not limited to shapes, sizes, and positional relationships which are exemplified in each drawing.

### First Embodiment

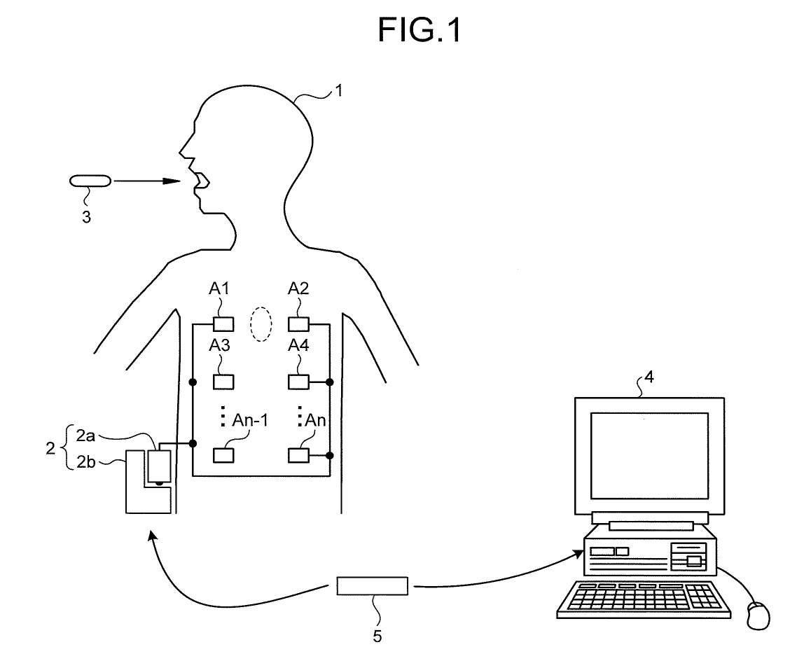

FIG. 1 is a schematic diagram illustrating an overall configuration of a body-insertable apparatus system according to a first embodiment of the invention. The body-insertable apparatus system uses a capsule endoscope as an example of a body-insertable apparatus. As shown in FIG. 1, the body-insertable apparatus system includes a capsule endoscope 3 that is introduced into a subject 1 to image images of the inside of a body cavity to thereby perform data transmission of an image signal or the like with respect to a transmitting and receiving apparatus 2 in a wireless manner, the transmitting and receiving apparatus 2 that receives image data of the inside of the body cavity, which has been transmitted from the capsule endoscope 3 in the wireless manner, a workstation 4 that displays the images of the inside of the body cavity based on an image signal received by the transmitting and receiving apparatus 2 so that the displayed images can be subjected to analysis processing or the like, and a portable recording medium 5 that delivers data between the transmitting and receiving apparatus 2 and the workstation 4.

The transmitting and receiving apparatus 2 includes an antenna unit 2a that has a plurality of reception antennas A1 to An which are affixed to an external surface of the subject 1, and a reception main body unit 2b that performs a process or the like of a wireless signal received through the plurality of reception antennas A1 to An. The antenna unit 2a and the reception main body unit 2b are connected to be attachable to and detachable from each other through a connection portion such as a connector. In addition, each of the reception antennas A1 to An is equipped in a jacket that is wearable by the subject 1, so that the subject 1 may mount the reception antennas A1 to An by wearing the jacket. In addition, in this case, the reception antennas A1 to An may be attachable to and detachable from the jacket.

The workstation 4 is to display the images of the inside of the body cavity imaged by the capsule endoscope 3, and performs image display based on data which is obtainable by the portable recording medium 5. Specifically, the workstation 4 may have a configuration of directly displaying images on a liquid crystal display or the like, or a configuration of outputting images to another medium such as a printer or the like.

CompactFlash (registered trademark) or the like is used as the portable recording medium 5, and the portable recording medium 5 may have functions capable of being attachable to and detachable from the reception main body unit 2b and the workstation 4, and capable of outputting and recording information at the time of insertion to both the reception main body unit 2b and the workstation 4. Specifically, the portable recording medium 5 is inserted into the reception main body unit 2b while the capsule endoscope 3 is moved inside the body cavity of the subject 1, and thereby data transmitted from the capsule endoscope 3 is recorded on the portable recording medium 5. After the capsule endoscope 3 is excreted from the subject 1, that is, after the imaging of the inside of the subject 1 is finished, the imaged data is taken out from the reception main body unit 2b and inserted into the workstation 4, and thereby the recorded data is read by the workstation 4.

Data delivery between the reception main body unit 2b and the workstation 4 is performed by the portable recording medium 5, and thereby it is possible to allow the subject 1 to freely act while imaging the inside of the body cavity, and to reduce a data delivery period between the reception main body unit 2b and the workstation 4. Incidentally, the data delivery between the reception main body unit 2b and the workstation 4 may be performed in such a manner that the reception main body unit 2b is connected to the workstation 4 in a wired or wireless manner using another built-in recording device.

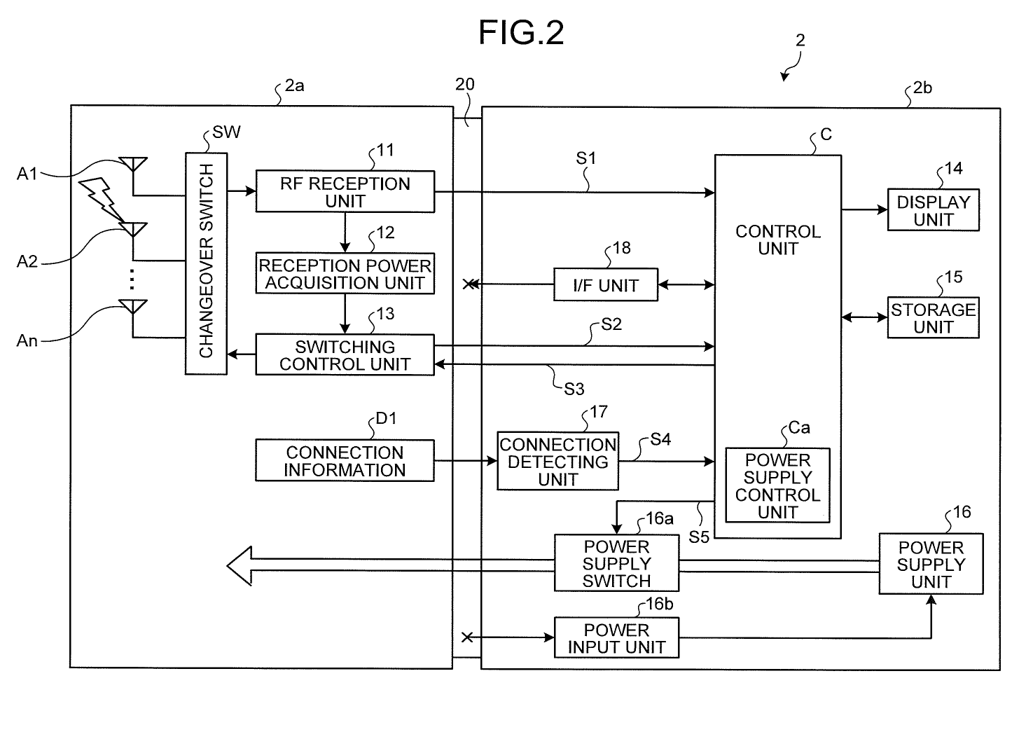

Here, with reference to FIG. 2, the antenna unit 2a and the reception main body unit 2b will be described in detail. FIG. 2 is a block diagram illustrating a configuration of the transmitting and receiving apparatus 2. As shown in FIG. 2, the antenna unit 2a and the reception main body unit 2b are connected by a connector 20 as a connection portion realized by a USB connector or the like.

The antenna unit 2a receives wireless signals transmitted from the capsule endoscope 3 to amplify the received wireless signals, and demodulates the amplified signals. As shown in FIG. 2, the antenna unit 2a includes a changeover switch SW that performs connection changeover processing for selectively switching any one of the reception antennas A1 to An, an RF reception unit 11 that is connected to the post-stage of the changeover switch SW, amplifies wireless signals from the reception antennas A1 to An which are switched/connected by the changeover switch SW and demodulates the amplified signals, a reception power acquisition unit 12 that acquires power information (intensity) of the signal received by the RF reception unit 11, and a switching control unit 13 that performs switching of the reception antennas A1 to An based on the power information acquired by the reception power acquisition unit 12.

The reception main body unit 2b receives and processes the demodulated signal demodulated by the antenna unit 2a. As shown in FIG. 2, the reception main body unit 2b includes a display unit 14 that displays information such as a connection state, a storage unit 15 that stores a variety of information, a power supply unit 16 that supplies power to the reception main body unit 2b and the antenna unit 2a, a power supply switch 16a that turns power supply to a connection destination such as the antenna unit 2a on/off, a connection detecting unit 17 that receives connection information D1 from the antenna unit 2a and detects presence and absence of connection with the antenna unit 2a or the like, an interface (I/F) unit 18 that performs input/output of a variety of information from the outside other than the antenna unit 2a, and a control unit C that controls each of these components. Power input from the outside to a power input unit 16b through the connector 20 is accumulated and charged in the power supply unit 16.

In the transmitting and receiving apparatus 2, the RF reception unit 11 amplifies wireless signals including the image data or the like output from the changeover switch SW, and outputs demodulated signals S1 to the control unit C and outputs a signal of reception power indicating a signal intensity of the amplified wireless signals to the reception power acquisition unit 12. The demodulated signals S1 input from the antenna unit 2a are subjected to signal processing by the control unit C, and then stored in the storage unit 15. A reception intensity signal output to the reception power acquisition unit 12 is imported to the switching control unit 13. The switching control unit 13 outputs reception intensity signals S2 to the control unit C to thereby receive a control signal. The control unit C selects, as the reception antenna for acquiring image data, the reception antenna received with the largest signal intensity, based on each of the reception intensity signals S2 obtained by sequentially switching the reception antennas A1 to An, and outputs a switching signal S3 for instructing switching to the reception antenna to the switching control unit 13. The switching control unit 13 switches to a corresponding reception antenna based on the switching signal S3.

The control unit C includes a power supply control unit Ca that controls power supply to the antenna unit 2a. The power supply control unit Ca performs a control for supplying power from the power supply unit 16 to the antenna unit 2a by outputting an instruction signal S5 for allowing the power supply switch 16a to be turned on, based on a connection detecting signal S4 from the connection detecting unit 17 which detects that the antenna unit 2a and the reception main body unit 2b are connected. Incidentally, the control unit C may display a connection state to the display unit 14 including LED or the like and control the displayed connection state, based on the connection detecting signal S4 sent from the connection detecting unit 17. For example, a red LED is lit in a non-connected state, and a green LED is lit in a connected state.

In addition, the control unit C stores signal intensity received in each of the reception antennas together with image data in the storage unit 15 in association with the selected reception antenna. Incidentally, the stored signal intensity of each of the reception antennas is used as information for calculating a position of the capsule endoscope 3 inside the body when the image data is received.

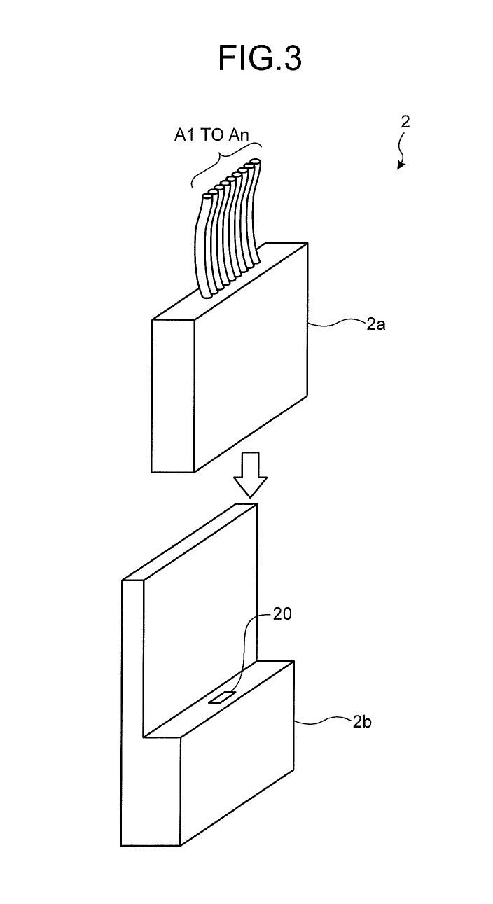

Here, with reference to FIG. 3, an external configuration of the transmitting and receiving apparatus 2 will be described in detail. FIG. 3 is a perspective diagram schematically illustrating an overall configuration of the transmitting and receiving apparatus 2 in a state in which the antenna unit 2a and the reception main body unit 2b are separated. The transmitting and receiving apparatus 2 includes the antenna unit 2a and the reception main body unit 2b that are attachable and detachable in a vertical direction of FIG. 3.

The reception antennas A1 to An are connected to an upper surface portion of the antenna unit 2a. Meanwhile, the reception main body unit 2b includes the connector 20 formed in a center portion of a surface facing the antenna unit 2a in an insertion/detachment direction of the antenna unit 2a from among two surfaces facing the antenna unit 2a at the time of mounting the antenna unit 2a.

The connector 20 of the reception main body unit 2b is fitted to a connector (not shown) that the antenna unit 2a has, and connects corresponding all signal lines and power supply lines between the antenna unit 2a and the reception main body unit 2b. In this instance, since the connector 20 is covered by the antenna unit 2a, another connection target cannot be connected to the connector 20.

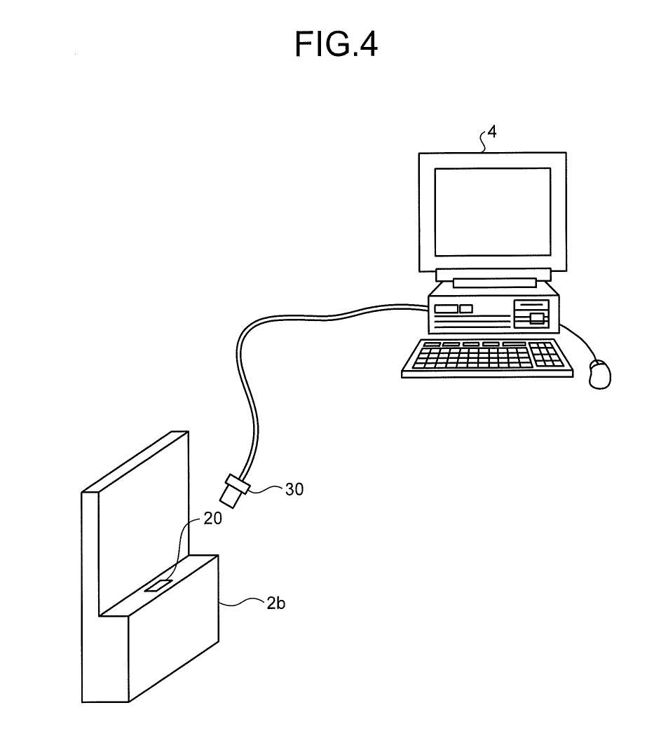

In addition, FIG. 4 is a schematic diagram illustrating a configuration of a main portion of the body-insertable apparatus system. As shown in FIG. 4, the reception main body unit 2b is connected to the workstation 4 through the connector 20 and a connector 30 of the workstation 4. In this instance, the connector 30 of the workstation 4 side is connected to the connector 20 of the reception main body unit 2b. Here, the connection detecting unit 17 detects and determines signals (connection information) indicating whether the connection destination is the antenna unit 2a or the workstation 4.

According to the first embodiment described above, the connector that is connected with the antenna unit and the workstation is a common connector, so that communication with the antenna unit and the workstation may be exclusively and reliably performed because of a configuration in which, when one of the antenna unit and the workstation is connected with the connector, the other is not connected with the connector. In addition, on the reception main body unit side, only a control for confirming a connection target may be performed, and therefore it is unnecessary to perform signal switching or the like corresponding to a process, and the configuration can be simplified.

In addition, in the conventional transmitting and receiving apparatus, it is necessary to perform a waterproofing process with respect to an external output connector such as a rubber cap or the like, but the transmitting and receiving apparatus according to the invention may be used without performing the waterproofing process because it is kept watertight due to close contact between the antenna unit and the reception main body unit at the time of use such as when mounting the antenna unit, and thus the configuration can be further simplified.

Incidentally, the connector of the reception main body unit may be disposed in any position as long as the connector is on a surface of the reception main body unit, the surface being covered by the antenna unit when the antenna unit is mounted.

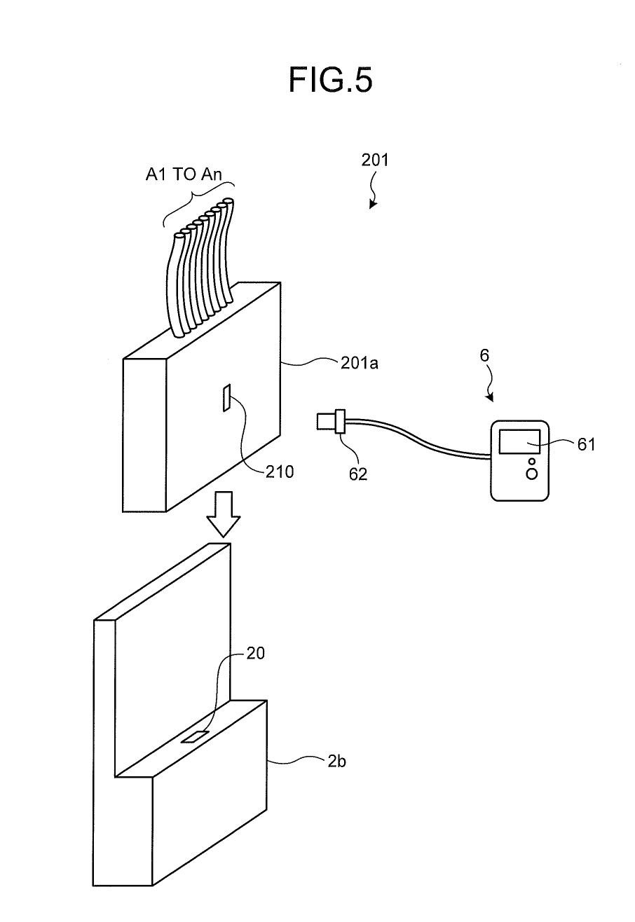

FIG. 5 is a schematic diagram illustrating a configuration of a main portion of a body-insertable apparatus system according to a modified example 1 of the first embodiment of the invention. In a transmitting and receiving apparatus 201 shown in FIG. 5, the transmitting and receiving apparatus 2 is connected with a viewer 6 as a display device that displays, in a display unit 61, an image imaged by the capsule endoscope 3 based on electrical signals output from the transmitting and receiving apparatus 201. The transmitting and receiving apparatus 201 and the viewer 6 are connected by a connector 62 that the viewer 6 has and a connector 210 that is mounted on a surface not opposing to the transmitting and receiving apparatus 201 of an antenna unit 201a.

The viewer 6 is a portable viewer that is formed to have a size of a degree that the viewer 6 can be held by the hand of an operator, and has a function of displaying images based on the electrical signals output from the transmitting and receiving apparatus 201. To realize the function, the viewer 6 includes the display unit 61 using a small LCD for image display. The viewer 6 includes the connector 62 capable of being connected with the connector 210 of the antenna unit 201a.

The connector 62 can be connected with the connector 210, and cannot be connected with the connector 20 of the reception main body unit 2b. In addition, it is preferable that the connector 210 has a different kind of shape or the like from that of the connector 20.

The image data received by the antenna unit 201a is output to the control unit C of the reception main body unit 2b. The image data input to the control unit C is again output to the antenna unit 201a, and output to the viewer 6 through the connector 210.

According to the modified example 1 of the first embodiment of the invention described above, only when the antenna unit is connected to the reception main body unit, image display by the viewer is possible. In particular, when the reception main body unit is connected with the workstation, the reception main body unit and the viewer are never connected, and therefore the workstation can exclusively and effectively perform download of data from the transmitting and receiving apparatus. In addition, by providing a connector of the viewer in the antenna unit, it is possible to prevent unnecessary connection without being connected to the reception main body unit in a state in which the antenna unit is not connected to the reception main body unit.

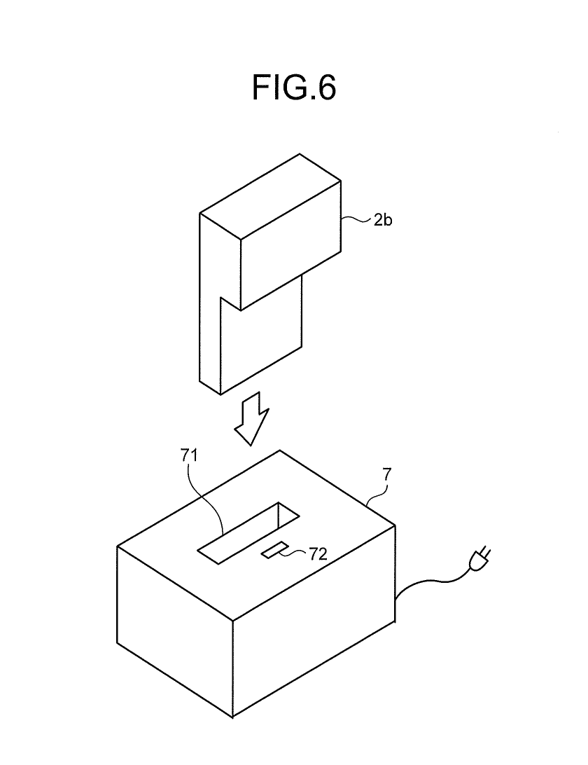

FIG. 6 is a perspective diagram schematically illustrating a configuration of a main portion of a body-insertable apparatus system according to a modified example 2 of the first embodiment of the invention. The body-insertable apparatus system shown in FIG. 6 has a configuration of a charger 7 for charging the reception main body unit 2b. As shown in FIG. 6, the charger 7 includes a holding hole 71, through which a part of the reception main body unit 2b is inserted to thereby hold the part of the reception main body unit 2b, and a connector 72 that is connected with the connector 20 of the reception main body unit 2b to thereby be electrically conducted with the reception main body unit 2b.

The connector 20 of the reception main body unit 2b is connected with the connector 72, and charges the power supply unit 16 through the power input unit 16b. In this instance, since the connector 72 connects the connector 20 of the reception main body unit 2b, the antenna unit 2a, the workstation 4, and the viewer 6 are never connected with the reception main body unit 2b.

According to the modified example 2 of the first embodiment described above, it is possible to exclusively charge the reception main body unit. In addition, since the reception main body unit, the workstation, or the like is never connected during the charging, it is possible to efficiently charge the reception main body unit, and at the same time, since information signals acquired from a patient and commercial power supply signals are never simultaneously connected, it is possible to ensure safety of the patient and at the same time simplify a configuration of the reception main body unit and/or the charger.

### Second Embodiment

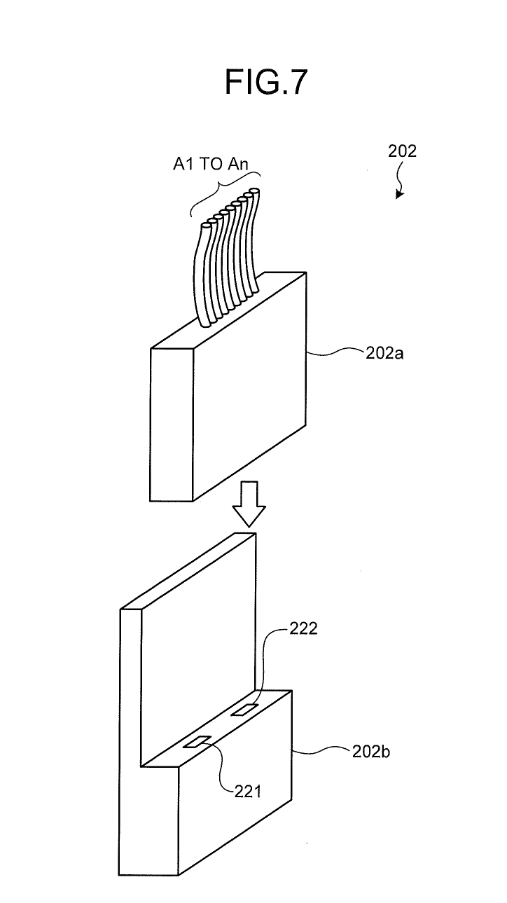

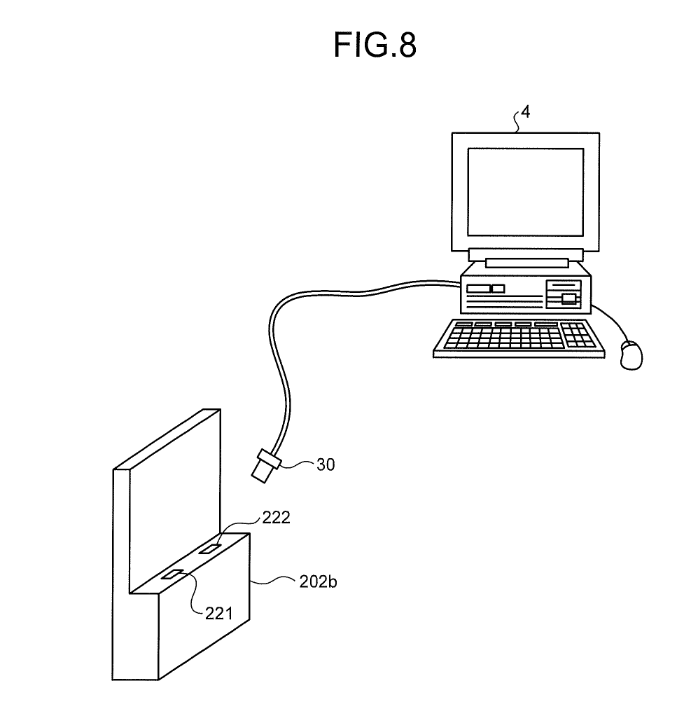

FIG. 7 is a perspective diagram schematically illustrating a configuration of a transmitting and receiving apparatus of a body-insertable apparatus system according to a second embodiment of the invention. FIG. 8 is a schematic diagram illustrating a configuration of a main portion of the body-insertable apparatus system according to the second embodiment of the invention. In a transmitting and receiving apparatus 202 shown in FIGS. 7 and 8, a reception main body unit 202b is respectively connected with an antenna unit 202a and the workstation 4, and includes connectors 221 and 222 which are juxtaposed on the same surface.

The antenna unit 202a includes a connector, which is not shown, formed on a surface opposing a surface on which the reception antennas A1 to An are provided. The antenna unit 202a is connected with the reception main body unit 202b by coupling the connector of the antenna unit 202a and the connector 221.

The workstation 4 is connected with the reception main body unit 202b by being coupled with the connector 222, by the above-described connector 30. Incidentally, the connector 221 is connected with the connector of the antenna unit 202a, and the connector 222 is connected with the connector 30. When the connector 221 is connectable with the connector of the antenna unit 202a, the connector 221 cannot be connected with the connector 30. Meanwhile, when the connector 222 is connectable with the connector 30, the connector 222 cannot be connected with the connector of the antenna unit 202a. The connectors 221 and 222 have different kinds of shapes or the like. Incidentally, any form that corresponds to a connector of a connection target may be applied.

According to the second embodiment described above, it is possible to exclusively perform communication between the antenna unit and the workstation because the connector that is connected with each of the antenna unit and the workstation is individually provided, and at the same time, when one of the antenna unit and the workstation is connected with the connector, the other is not connected with the connector. In addition, it is possible to further simplify a configuration of the reception main body unit without performing control of confirming the connection target.

Incidentally, in the second embodiment, an example in which the reception main body unit includes two connectors in which one of the two connectors is connected with the antenna unit and the other is connected with the workstation has been described, but at least three connectors may be disposed by separately providing a connector for charge or the like.

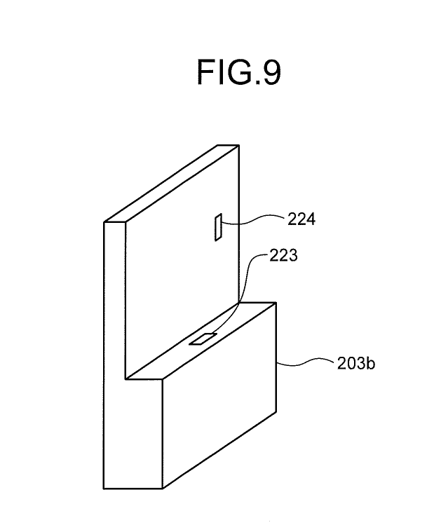

FIG. 9 is a perspective diagram schematically illustrating a configuration of a main portion of a body-insertable apparatus system according to a modified example of the second embodiment of the invention. As shown in FIG. 9, a reception main body unit 203b includes connectors 223 and 224 which are respectively connected with the antenna unit 202a and the workstation 4 which are shown in FIGS. 7 and 8.

The connector 223 is formed on a surface perpendicular to an insertion/detachment direction of the antenna unit 202a, and is coupled with the connector of the antenna unit 202a to be electrically connected. In addition, the connector 224 is formed on a surface parallel to the insertion/detachment direction of the antenna unit 202a, and is coupled with the connector 30 of the workstation 4 to be electrically connected.

According to the above-described modified example, it is possible to exclusively perform communication between the antenna unit and the workstation because the connector that is connected with each of the antenna units and the workstation is individually provided, and at the same time, when one of the antenna units and the workstation is connected with the connector, the other is not connected with the connector. In addition, since the connector is respectively provided on a different surface, it is possible to easily identify each connector visually, and to more reliably connect each connector.

As above, the transmitting and receiving apparatus and the body-insertable apparatus system according to the invention is useful when exclusively and reliably performing transmission/reception of signals such as information or the like.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A transmitting and receiving apparatus that is connected with each of a plurality of connection targets, which include an antenna unit and a charger, to perform transmission/reception of a signal, comprising:
a connection portion that is provided on a surface of the transmitting and receiving apparatus, connectable with the plurality of connection targets, and is connected, at one time, with only one connection target to prevent a malfunction.

2. The transmitting and receiving apparatus according to claim 1, comprising a connection detecting unit that detects a signal of the connected connection target,
wherein the connection portion is a single connector.

3. The transmitting and receiving apparatus according to claim 1, wherein the connection portion includes a plurality of connectors corresponding to the connection targets, respectively.

4. The transmitting and receiving apparatus according to claim 3, wherein the plurality of connectors are arranged on the same surface.

5. The transmitting and receiving apparatus according to claim 1, wherein the connection portion includes
a first connector that is attachable to and detachable from the antenna unit, and
a second connector that is provided on a surface, which is covered by the antenna unit when the antenna unit is mounted, and connectable with another connection target.

6. An antenna unit that is connectable with a plurality of connection targets, which include a charger, includes a connection portion that is connected, at one time, with only one connection target to prevent a malfunction, and is connectable with a transmitting and receiving apparatus that performs transmission/reception of a signal, the antenna unit comprising:
a connector that is connectable with a display device for displaying image data received by the antenna unit, the connector being provided on a surface of the antenna unit that does not oppose to the transmitting and receiving apparatus when the antenna unit is mounted on the transmitting and receiving apparatus.

7. A body-insertable apparatus system comprising:
a body-insertable apparatus that is introduced into a subject and moves inside the subject; and
a transmitting and receiving apparatus that transmits/receives a signal to/from another connection target including the body-insertable apparatus and a charger,
wherein the transmitting and receiving apparatus includes an antenna unit that receives information from the body-insertable apparatus in a wired or wireless manner, and a reception main body unit that includes a first connection portion that is attachable to and detachable from the antenna unit and a second connection portion that is provided on a surface, which is covered by the antenna unit when the antenna unit is mounted, and is connectable with another connection target including the charger.

8. The system introduced into the subject according to claim 7, wherein each of the first connection portion and the second connection portion is a single connector and includes a connection detecting unit that detects a signal of the antenna unit or the connection target which is connected.

9. The system introduced into the subject according to claim 7, wherein the antenna unit includes a third connector that is connectable with a display device for displaying image data received by the antenna unit, the third connector being provided on a surface of the antenna unit that does not oppose to the reception main body unit when the antenna unit is mounted on the reception main body unit.
